(19) United States
(12) Patent Application Publication
(10) Pub. No.: US 2023/0168217 A1
(43) Pub. Date: Jun. 1, 2023

(54) Test strip for the detection of neutral analytes in a sample

(71) Applicant: Fepod Oy Ltd, Helsinki (FI)

(72) Inventors: Niklas Wester, Aalto (FI); Jari Koskinen, Aalto (FI); Ilkka Varjos, Espoo (FI); Björn Mikladal, Helsinki (FI)

(21) Appl. No.: 17/921,640

(22) PCT Filed: Apr. 27, 2021
(86) PCT No.: PCT/FI2021/050312

§ 371 Date: Oct. 27, 2022

(30) Foreign Application Priority Data
Apr. 27, 2020 (FI) 20205422

(51) Int. Cl. G01N 27/327; G01N 27/333; G01N 33/543

(52) U.S. Cl. CPC G01N 27/3272; G01N 27/3335; G01N 33/5438

(57) ABSTRACT

According to an example aspect of the present invention, there is provided a disposable multilayer test strip comprising a substrate onto which is deposited an electrode assembly comprising a carbon-based working electrode, a carbon-based counter electrode, a pseudoreference electrode, wherein the pseudo reference electrode, the working electrode and the counter electrode, are arranged adjacent to each other in the same plane, contacts for contacting the electrodes directly to a voltage supply, and the test strip further comprises a permselective membrane layer. The electrodes of the electrode assembly layer are electrically separated from one another and said electrode assembly layer is positioned between the substrate and the permselective membrane layer. The permselective membrane has a structure adapted to allow passage of one or more electronically neutral analytes in a sample to be analysed across the permselective membrane to the electrode assembly.

### FIELD

The present invention relates to a multilayer test strip, particularly a multilayer test strip for the detection of neutral analytes, such as paracetamol in a sample and a method of manufacturing such a multilayer test strip. Further, the invention relates to a system for the detection of neutral analytes comprising a multilayer test strip and a measurement circuit. Moreover, the present invention relates to a method for the measurement of neutral analytes in sample. Furthermore, the present invention relates to a method of diagnosing overdose and/or toxic levels of a neutral analyte or substance such as paracetamol in a patient. Still further the present invention relates to determining individual pharmacokinetic parameters with the aim of personalized treatment.

### BACKGROUND

Paracetamol, otherwise known as acetaminophen, is one of the most widely used analgesics with antipyretic properties. It is readily available, inexpensive and is better tolerated than NSAIDs, and is therefore widely recommended as the first choice for treatment of a wide range of pain. Unlike NSAIDs large doses of paracetamol can cause hepatotoxicity. Paracetamol is one of the most commonly taken drugs in overdose and paracetamol poisoning is currently the leading cause of acute liver failure in the United States and Europe. In the United stated alone, there are >111 000 exposures reported to the poison center and 40 000 associated emergency department cases annually. Both intentional and unintentional exposures to toxic levels of paracetamol are common.

The toxicity of paracetamol is due to the highly reactive metabolite N-acetyl-p-benzoquinone imine (NAPQI). Toxic doses of paracetamol cause more of the drug to be metabolized by the CYP2E1 enzyme, into NAPQI. At therapeutic doses this toxic metabolite is immediately inactivated by conjugation with glutathione and excreted trough urine. At toxic concentrations, however, this route of detoxification is depleted. NAPQI can bind covalently to cellular proteins and form toxic adducts, which may cause mitochondrial dysfunction and early oxidant stress. This may ultimately lead to liver cell necrosis and acute liver failure. The cellular damage has been found to be directly related to the dose of paracetamol.

Paracetamol poisoning can be effectively treated with the glutathione precursor N-acetylcysteine. Unfortunately paracetamol poisoning shows few and nonspecific symptoms in the first 24 hours. Furthermore, the N-acetylcysteine treatment is most effective when initiated within 8-12 h after exposure and after 15 h the efficacy of the antidote rapidly diminishes. For these reasons, the National Academy of Clinical Biochemistry has endorsed screening for paracetamol in all emergency department patients who present with intentional drug ingestion. Diagnosis of paracetamol overdose is usually carried out by determining the paracetamol serum concentration. The Rumack- Matthew nomogram that plots the paracetamol concentration as a function of time post-ingestion, is helpful in determining the likelihood of hepatotoxicity. Serum levels at or above 200 µg/ml (1.323 mM) at 4 hours postingestion and 6.25 µg/mL (43.1 µM) at 24 h post-ingestion have been found to consistently predict hepatotoxicity. The line between these points is referred to as the probable toxicity line. The FDA later required the addition of an additional line 25% below the original line, to build in some additional safety.

### SUMMARY OF THE INVENTION

In clinical settings rapid tests are usually carried out with spectrophotometric methods because of relative simplicity and low cost. Despite these advantages, this method is still confined to specialized laboratories and is poorly suited for point-of-care testing. Moreover, interference causing both falsely high and low results has been reported with these methods. In addition, competitive lateral flow immunoassays are also available for qualitative determination of paracetamol. These tests are, however not quantitative and due to high cut-off concentrations false negatives have been reported. Therefore, the development of a highly mobile, simple and quantitative point-of-care assay for screening of paracetamol poisoning is highly desirable.

The invention is defined by the features of the independent claims. Some specific embodiments are defined in the dependent claims.

According to a first aspect of the present invention, there is provided a disposable multilayer test strip comprising a substrate onto which is deposited an electrode assembly comprising a carbon-based working electrode, a carbon-based counter electrode and a pseudoreference electrode. The pseudoreference electrode, the working electrode and the counter electrode, are arranged adjacent to each other in the same plane. The strip further comprises contacts for contacting the electrodes directly to a voltage supply, as well as a permselective membrane layer. The electrodes of the electrode assembly layer are electrically separated from one another and the electrode assembly layer is positioned between the substrate and the permselective membrane layer. The permselective membrane has a structure adapted to allow passage of one or more electronically neutral analytes in a sample to be analysed across the permselective membrane to the electrode assembly.

According to second aspect of the present invention, there is provided an apparatus comprising a memory configured to store reference data, at least one processing core configured to process information from a multilayer test strip described herein, compare the information from the strip described herein to the reference data; and draw conclusions on the information processed from the strip described herein.

According to a third aspect of the present invention, there is provided a method for detecting electronically neutral analytes in a sample comprising the steps of providing a sample, contacting the sample electrically with a working electrode and a counter electrode of an electrode assembly of a multilayer test strip, changing voltage between the working electrode and counter electrode, measuring a current between the working electrode and counter electrode as relation to the voltage applied between the working electrode and counter electrode and detecting a change in current characteristic of one or more analytes in the sample.

According to a fourth aspect of the present invention, there is provided a method of diagnosing overdose in a patient. The method comprises obtaining a sample from a subject, contacting the sample electrically with a working electrode and a counter electrode of an electrode assembly of a multilayer test strip, changing voltage between the working electrode and counter electrode, measuring a current between the working electrode and counter electrode as relation to the voltage applied between the working electrode and counter electrode, detecting a change in current characteristic of one or more analytes in the sample, determining the amount of analyte in the sample in an apparatus according to the second aspect of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates one example for a process of fabricating multilayer test strips in accordance with at least some embodiments of the present invention;

FIG. 2 shows scanning electron micrographs of cross sections of a Nafion coated working electrode (A) and Nafion coated reference electrode (B) in accordance with at least some embodiments of the present invention.

FIG. 3 comprises three graphs showing (A) the potential of an uncoated and nafion coated pseudo-reference electrode against Ag/AgCl(sat.) in 0.1 M PBS solution, showing (B) potential as function of the Cl⁻ concentration in KCl solutions and showing (C) a cyclic voltammogram of 1 mM Ru(NH₃)₆ in 1 M KCl in accordance with at least some embodiments of the present invention. All measurements carried out in a conventional 50 ml electrochemical cell.

FIG. 4 shows (A) Comparison of DPV measurements carried out in 50 µM PA in a 50 mL cell and with a 40 µL drop, and (B) Optimization of DPV pulse amplitude for 50 µM PA in 40 µL diluted human plasma.

FIG. 5 shows DPVs of increasing concentrations of paracetamol in (A) PBS, (B) human plasma (C) whole blood. (D) shows the linearization of results in all measured matrices. The error bars show the standard deviations of 4 measurements with different electrodes.

FIG. 6 shows (A) CV of 1 mM Ru(NH₃)₆ in PBS and plasma and (B) DPV peak currents as a function of scan number in 1 mM PA in whole blood and plasma.

FIG. 7 illustrates an interference study. (A) DPV scans in blank PBS (black line), interferent alone (blue line) and interferent + 50 µM PA (red line). (B) The background subtracted peak current for 50 µM PA alone (red) and PA in the presence of interferent (blue). The error bar represents a 5% error defined as the tolerance limit. The DPV scans in (A) have been offset for clarity.

### EMBODIMENTS

The present invention relates to a disposable electrochemical test strip for quantitative point of care determination of molecules of interest, namely neutral analytes, that have been overdosed or otherwise administered to or accumulated in a subject at toxic or therapeutic levels. The present invention further relates to a method of producing such test strips. With the process of fabrication or manufacturing according to the present invention, highly conductive, well electrically isolated and patterned carbon-based electrodes are printed on substrates. By means of the present invention a screen printed silver pseudo-reference electrode with excellent shelf life, with long term stability and short hydration times is produced. With this test strip low enough detection limits and wide enough linear range for determination of molecules of interest, e.g. paracetamol concentration in suspected paracetamol poisoning is achieved. It has surprisingly been found that detection and quantitative determination of molecules of interest e.g. paracetamol can be carried out with an assay according to embodiments of the present invention with a sample of only 20 µL in volume, said sample comprising e.g. a finger-prick sample of blood optionally diluted with up to 20 µL PBS, venous blood , or urine or venous blood, optionally diluted with PBS, or even saliva. No further sample treatment is required and fast results are obtained, an assay time of less than 5 minutes is achieved, which is extremely important in cases of overdose and toxicity. Moreover, selectivity is also achieved in the presence of several interferents.

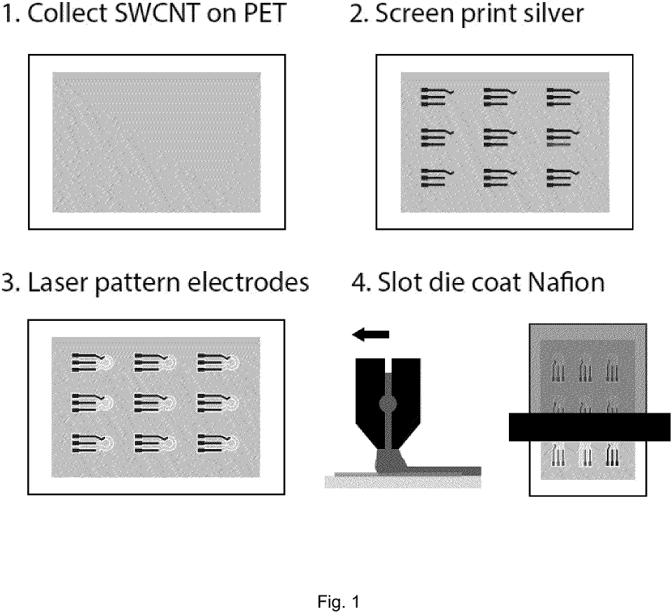

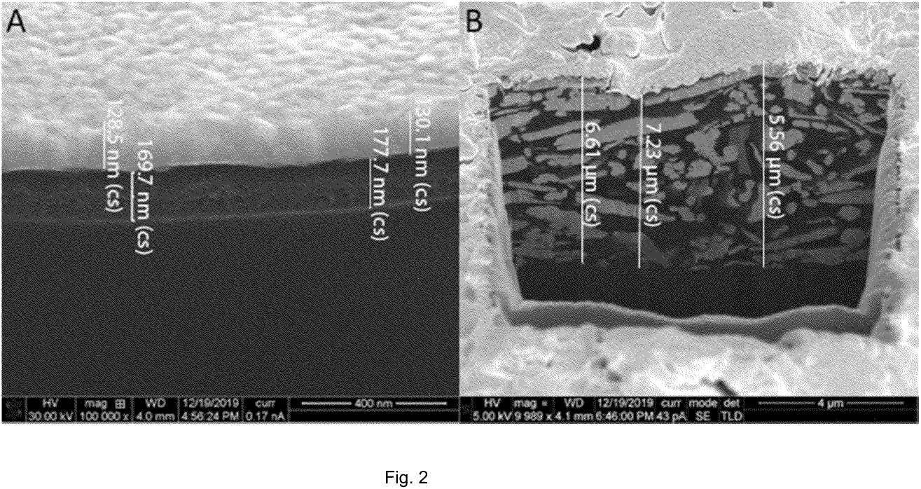

FIG. 1 illustrates the production of sensor strips. In this exemplary embodiment SWCNTs were first grown by aerosol CVD and collected on a filter. The SWCNT network was then press-transferred onto an A4 PET sheet and densified by spraying IPA from a spray bottle and dried with nitrogen, e.g. blow dried or dried with compressed nitrogen. To realize patterned electrodes, lines separating the electrodes were ablated. To realize a reference electrode silver lines were screen printed directly on top of the SWCNT layer (See FIG. 1, step 3). Silver contact pads were also fabricated in the same process. Finally, the whole A4 PET sheet was coated with Nafion in accordance with at least some embodiments of the present invention.

FIG. 2 shows the Cross-section images acquired from milled areas of A) the working electrode and B) the reference electrode in accordance with at least some embodiments of the invention. The overall thickness of the SWCNT/Nafion layer of the working electrode can be seen to be approximately 170 nm thick. A dark layer with a thickness of 65-75 nm between the SWCNT/Nafion layer and the Au coating can also be observed likely due to Nafion. This result is in agreement with previous studies, suggesting that the SWCNT are at least partially coated by Nafion. The cross-section of the Ag reference shows flat elongated Ag particles in the few µm in size range. Thicknesses between 5.9 to 7.2 µm were obtained for the cross-sections of the reference electrodes. Several measurements of the silver lines were also carried out with a contact profilometer giving thicknesses in the range of 5.5 to 7 µm. Due to the large roughness, a clear layer of Nafion cannot be discerned even on top of the Ag particles.

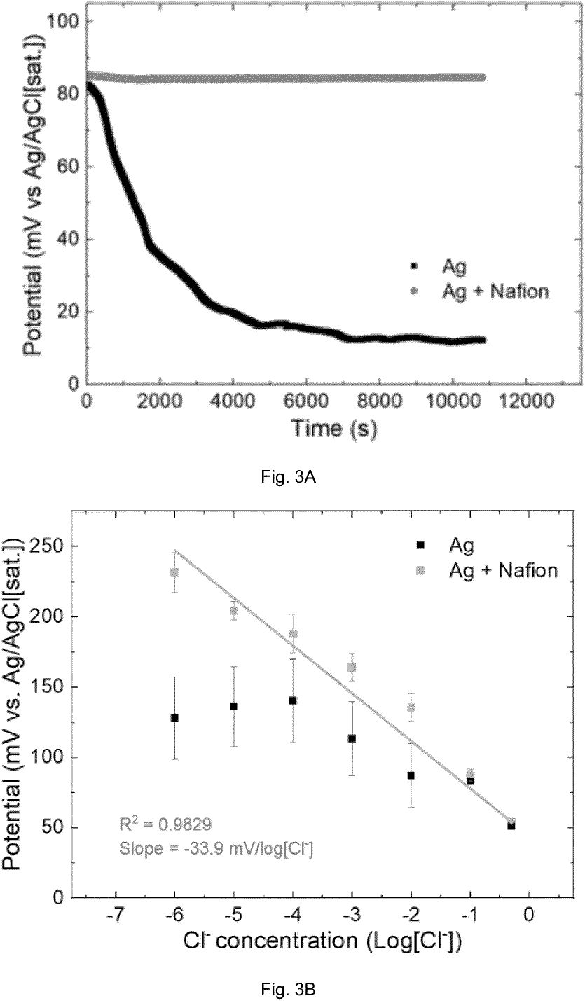

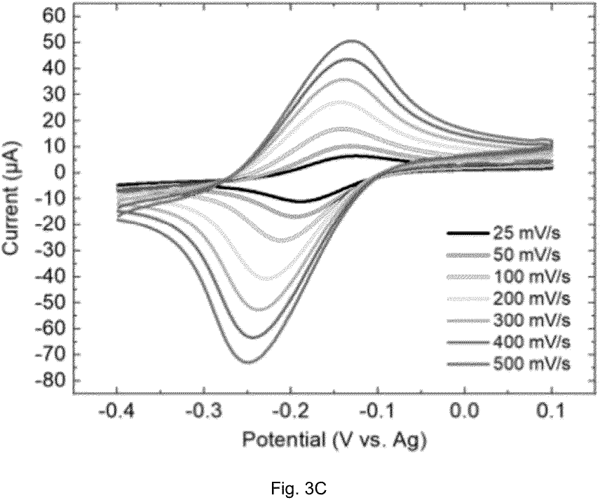

FIG. 3A shows the potential of the pseudoreference electrode vs an Ag/AgCl[sat] electrode of both the uncoated and Nafion coated screen printed Ag reference electrode in 0.1 M PBS solution supporting at least some embodiments of the invention. Both types of electrodes start at a potential of 84±1 mV. From FIG. 3A, it is, however, evident that the potential of the uncoated electrode drifts during the potential measurements.

FIG. 3B shows the potential of the Ag reference electrode as a function of the logarithm of the Cl⁻ concentration. The potential of the Nafion coated electrode depends linearly on the logarithm of the Cl⁻ concentration of the electrolyte with a slope of -33.9 mV/log[Cl⁻]. The potential of the uncoated Ag electrode also depends on the Cl⁻ concentration, but shows a less linear behavior. Despite the susceptibility toward Cl⁻ concentration, the Nafion coated electrode shows an immediately stable potential at all concentrations without any run-in time.

FIG. 3C shows the CV measurements with various scan rates in 1 mM Ru(NH₃)₆ in 1 M KCl. A peak potential separation (ΔEₚ) of 68.8 mV (scan rate: 100 mV/s) was obtained, indicating close to reversible electron transfer.

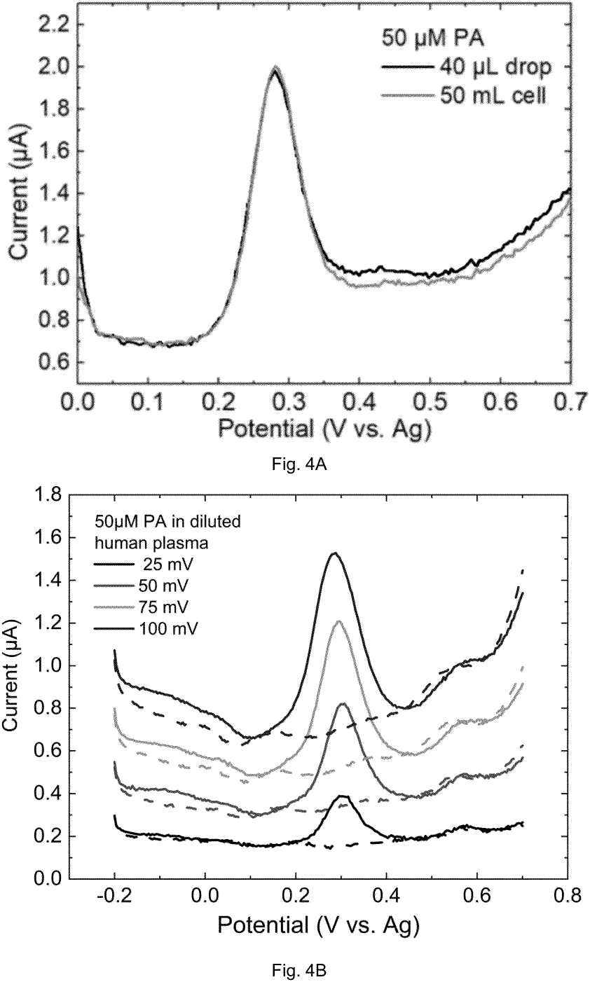

FIG. 4A shows DPV measurements carried out with the sensor strip in a conventional 50 ml electrochemical cell and with a 40 µL drop placed directly on the sensor. Background subtracted oxidation peaks of 1.178 and 1.159 µA (Average 1.07 µA in PBS conc. series) were measured for 50 µM PA in the 50 mL cell and the 40 µL drop, respectively.

FIG. 4B shows DPV measurements with different pulse amplitudes with a 40 µL drop diluted human plasma. It can be seen that a greater pulse amplitude expectedly leads to a larger sensitivity toward PA. Despite this only a negligible increase in the small peaks around 150 mV and 550 mV is observed with increase in pulse amplitude.

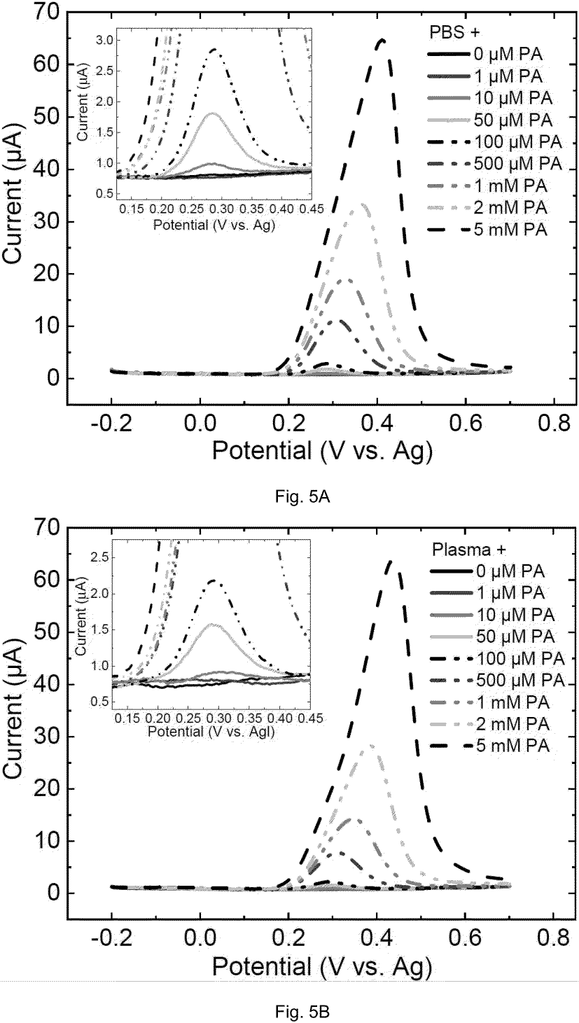

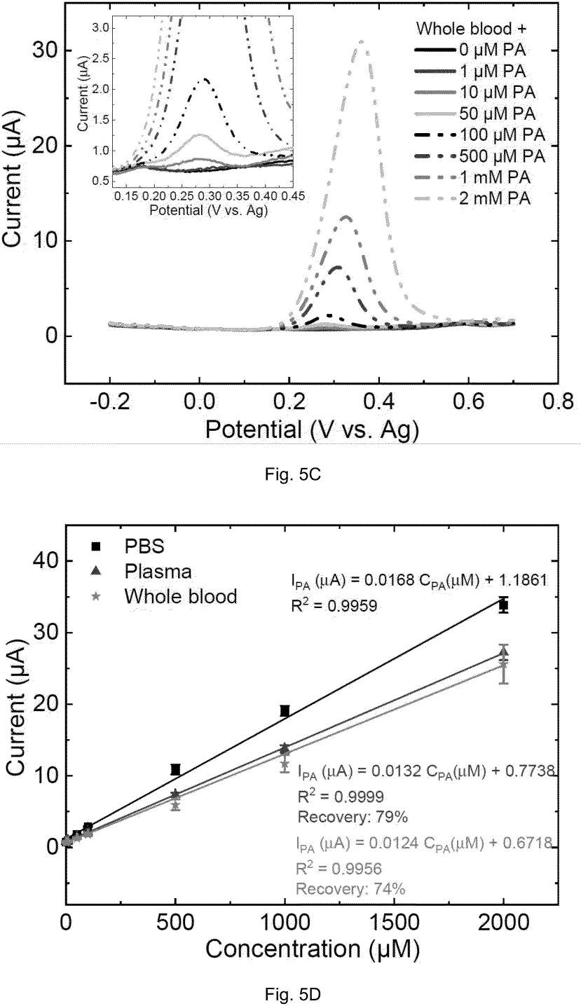

FIG. 5 shows the DPV measurements with increasing PA concentrations. It can be seen that the current scales linearly with the concentration in the concentration range of 1 µM to 2 mM.

FIG. 5D shows that recoveries of 79% and 74% were obtained in plasma and whole blood, respectively

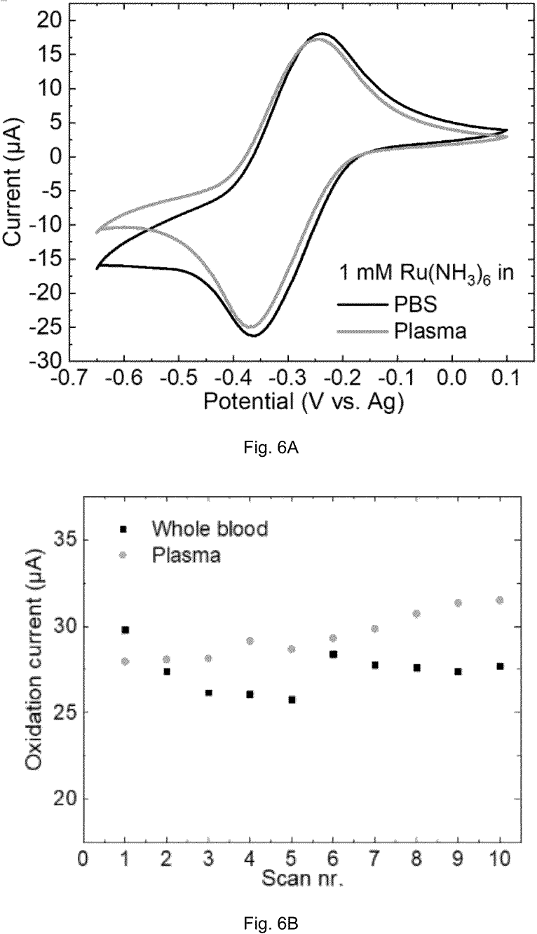

FIG. 6 shows no passivation of the electrode when 1 mM Ru(NH₃)₆ is measured in PBS and diluted human plasma.

FIG. 6B shows the measured oxidation currents as a function of scan number.

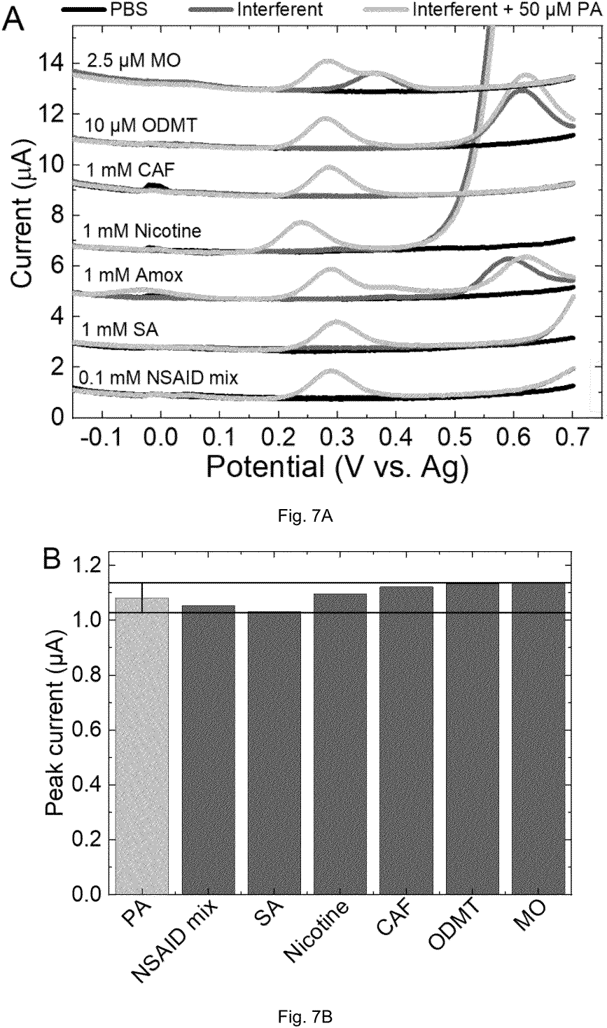

FIG. 7 shows the DPV scan in the absence and presence of a NSAID mix with 100 µM Ibuprofen, naproxen and aspirin, 1 mM salicylic acid (the metabolite of aspirin), 1 mM nicotine, 1 mM amoxicillin and 1 mM caffeine, as well as 2.5 µM morphine and 10 µM o-desmethyltramadol.

### DETAILED DESCRIPTION

As mentioned above the present invention relates to a multilayer test strip. In one embodiment is described a disposable multilayer test strip comprising a substrate onto which is deposited an electrode assembly. The electrode assembly comprises a carbon-based working electrode, a carbon-based counter electrode, and a pseudoreference electrode, wherein the pseudo reference electrode, the working electrode and the counter electrode, are arranged adjacent to each other in the same plane. The multilayer test strip comprises contacts for contacting the electrodes directly to a voltage supply, and the test strip further comprises a permselective membrane layer. The electrodes of the electrode assembly layer are electrically separated from one another and said electrode assembly layer is positioned between the substrate and the permselective membrane layer. By means of embodiments it has surprisingly been found that by adapting the structure of the permselective membrane, passage of analytes across the membrane can be controlled. Thus, in an embodiment the permselective membrane has a structure adapted to allow passage of one or more electronically neutral analytes in a sample to be analysed across the permselective membrane to the electrode assembly. For the purposes of embodiments electronically neutral analytes refers to analytes that are neutral under physiological conditions. In turn, physiological conditions means at a pH of approximately 7.4, e.g. the normal pH of human blood is typically in the range of 7.35 to 7.45. Zwitterions having an equal number of positive charges and negative charges are also included in the definition of neutral analytes.

In an embodiment the substrate of the strip is selected from the group consisting of polymer and glass. The substrates are selected based on the disposability. In an embodiment the substrate is a polymer such as polycarbonate or PET. Most preferably the substrate is polycarbonate since polycarbonate is biodegradable through the action of enzymes or by bacterial whole cells.

As described in embodiments above the strip comprises carbon-based electrodes. In one embodiment one or both of the carbon based electrodes comprises carbon selected from the group consisting of amorphous carbon, such as tetrahedral amorphous carbon, diamond-like carbon, graphite, carbon nanotubes, graphene and a mixture thereof. In a preferred embodiment one or both of the carbon based-electrodes comprises carbon nanotubes, in particular single-walled carbon nanotubes. Although each mentioned form of carbon is suitable in embodiments of the present invention, single-walled carbon nanotubes have a large surface area, high mechanical strength, high electrical conductivity and electrocatalytic activity, as well as having low charging current and enhanced mass transfer e.g. when networks/thin films deposited on insulating substrates, providing the added benefit of enabling a high signal-to-noise ratio in electrochemical detection. By means of aerosol chemical vapor deposition, large areas of porous SWCNT electrodes with high conductivity and surface area can be produced. This process allows for collection of patterned networks that can be easily press-transferred to produce electrodes without the need for modification of conventional carbon electrodes. This enables the production of inexpensive disposable SWCNT electrodes, on a wide range of substrates including polymers. SWCNT films can be patterned with standard lithography or laser patterned down to 10 µm by laser ablation without any damage to polymer substrates, including polycarbonate and PET. This process can be performed at high throughputs and is fully roll-to-roll compatible.

In a further embodiment the pseudo-reference electrode comprises silver. Ag/AgCl electrodes give satisfactory performance. Thus in one embodiment the pseudo-reference electrode comprises Ag/AgCl. However, it has surprisingly been found that the permselective membrane coating stabilizes the potential of the pseudo reference electrode so that the pseudo reference electrode can be fabricated in the same step as conductive silver lines eliminating the need for a second screen printing step with AgCl ink. Thus in a preferred embodiment the pseudo-reference electrode consists of silver.

The material of the permselective membrane may be selected from various materials. In an embodiment the permselective membrane comprises membrane material selected from the group of polymers consisting of Nafion, cellulose acetate, polyvinyl sulfonate, carboxymethyl cellulose, polylysine, overoxidised polypyrrole and other sulfonated polymers. In one embodiment the permselective membrane comprises conventional dialysis membrane material. Sulfonate groups in sulfonated polymers reject/repel negatively charged anions which interfere in the quantitative detection of neutral analytes such as paracetamol, while allowing neutral molecules to diffuse through the membrane. Thus sulfonated polymers are particularly desirable in embodiments of the present disposable multilayer test strip. Nafion, has a particularly high concentration of sulfonate groups throughout the polymer. Thus, in a preferred embodiment the membrane comprises Nafion. Nafion also has an affinity for cations such as morphine and tramadol as well as their metabolites, that often coexist in the samples and may also cause interference in the determination of neutral molecules, such as paracetamol. By means of embodiments it has been found that a nafion membrane functionalizes the electrode so that opioid intereferents such as e.g. morphine do not cause interference in the measurements of neutral analytes such as e.g. paracetamol.

In a further embodiment the structure of the permselective membrane is formed of one or more layers of membrane material applied to the strip, whereby a stack of membrane material layers forms the permselective membrane. By means of embodiments the thickness of the permselective membrane can thus be adapted.

It has been shown that permselective membranes, such as sulfonate containing polymers e.g. Nafion membranes form a coating that enriches cations due to ion-exchange reactions. Negatively charged channels in the coating, said channels having dimensions of a few nanometers do not allow the passage of anions. Neutral analytes may pass through the membrane by passive diffusion. Due to different interactions between different analytes and the permselective membrane such as sulfonate containing polymers membranes e.g. a Nafion membrane, different neutral molecules also exhibit different permeabilities. Thus in an embodiment, deposition parameters, such as deposition method, coating time, sulfonate group concentration in the membrane, e.g. Nafion membrane, number of layers, etc. are carefully controlled providing a multilayer test strip in which the permeability of neutrals and the degree of functionalization of the surface of the SWCNT electrodes can be controlled. We have previously shown that we can fabricate multilayer electrodes that optimize the enrichment of cations while blocking the anions and most of the neutrals from reaching the electrode, thus enabling selective detection of opioids in the presence of neutrals, such as paracetamol. The current multilayer electrode has been optimized, by controlling the deposition parameters to allow the passage of neutrals, without compromising the selectivity in measurements in complex matrices with high concentrations of anions, such as blood, urine and saliva. In the context of measuring paracetamol, the SWCNT electrode layer is also functionalized by the the permselective membrane in a way, such that the cations morphine and O-desmethyltramadol do not cause interference in the measurements at clinically relevant levels. In an embodiment the stack of membrane materials has a thickness in the range of 10 nm to 4000 nm. Stacking the layers has a dual effect. The amount of sulfonate groups increases as the number of layers increases, thus making the passage of cationic groups through the membrane increasingly difficult and as the layers are so thin, defects are found in each layer through which neutral analytes can easily pass. In an embodiment the permselective membrane has a thickness in the range of 50 to 3000 nm, preferably 75 to 2500 nm, suitably 100 to 2000 nm. In a further embodiment the permselective membrane has a thickness in the range of 50 to 400 nm, preferably 75 to 250 nm, suitably 100 to 200 nm.

Further embodiments relate to an apparatus for analysing data from a multilayer test strip. In an embodiment an apparatus comprises a memory configured to store reference data, at least one processing core configured to process information from a multilayer test strip according to embodiments described herein, compare the information from the strip according embodiments described herein to the reference data; and draw conclusions on the information processed from the strip according to embodiments described herein.

Further embodiments relate to a method for detecting electronically neutral analytes in a sample. In one embodiment, the method comprises the steps of providing a sample, contacting the sample electrically with a working electrode and counter electrode of an electrode assembly of a multilayer test strip, changing voltage between the working electrode and counter electrode, measuring a current between the working electrode and counter electrode in relation to the voltage applied between the working electrode and counter electrode and detecting a change in current characteristic of one or more analytes in the sample. In one embodiment the method detects free or unbound fractions neutral analytes in a sample. Free or unbound fractions are fractions that are not bound to blood and/or serum proteins. In a further embodiment, the detection of free or unbound fractions of neutral analytes is carried out without the use of equilibrium dialysis. In other words in a particular embodiment the detection of free or unbound fractions of neutral analytes is carried out in an equilibrium dialysis free detection method.

Further embodiments describe a method for detecting electronically neutral analytes in a sample. In an embodiment the method comprises providing a sample, typically the sample is a blood sample obtainable e.g. from a finger prick, contacting the sample electrically with a working electrode and counter electrode of an electrode assembly of a multilayer test strip described hereinabove, changing voltage between the working electrode and counter electrode, measuring a current between the working electrode and counter electrode in relation to the voltage applied between the working electrode and counter electrode and detecting a change in current characteristic of one or more analytes in the sample. In a further embodiment the electronically neutral analytes to be detected are selected from the group of paracetamol, tetrahydrocannabinol (THC), alprazolam, lorazepam, and general anesthetics such as propofol. In one embodiment the sample is diluted with a buffer solution, preferably with PBS. Preferably the sample is not diluted at all. In one embodiment the amount of sample contacted with the working electrode and counter electrode amounts to approximately 3.5 - 20 µl, preferably 5 - 15 µl, suitably 10 µl.

The voltage between the working electrode and the counter electrode is scanned according to the analytes to be detected, e.g. in an embodiment, the voltage between the working electrode and counter electrode is scanned from -0.2 V to 0.8 V at a scan rate, preferably from 0.1 V to 0.6 V, which are suitable ranges for the detection of paracetamol.

Similarly, in an embodiment the scan rate is adjusted according to the analytes to be detected. In an embodiment the scan rate is in the range of 5 to 1000 mV/s, preferably 10 - 400 mV/s.

Further embodiments describe the manufacturing process of a multilayer test strip. In an embodiment the method comprises the steps of providing an SWCNT network, pressing the SWCNT network onto a substrate, to form carbon-based electrodes, separating the electrodes by laser patterning, screen printing silver to form a silver pseudo reference electrode adjacent to a carbon-based working electrode and a carbon-based counter electrode, screen printing silver contact pads onto each electrode, coating the electrodes with a permselective membrane layer. In an embodiment the coating step is adapted to coat the electrodes with a predetermined thickness of permselective membrane. In an alternative embodiment carbon based electrodes are formed on the substrate from amorphous carbon . The amorphous carbon is applied onto the substrated by physical vapour depositions with shadow masks or by standard photolithography. By means of an embodiment of the method a multilayer test strip as described hereinabove is manufactured.

Also disclosed are embodiments in which overdose is diagnosed in a patient or subject. In one embodiment the method of diagnosis comprises obtaining a sample from a subject, contacting the sample electrically with a working electrode and a counter electrode of an electrode assembly of a multilayer test strip, changing voltage between the working electrode and counter electrode, measuring a current between the working electrode and counter electrode in relation to the voltage applied between the working electrode and counter electrode, detecting a change in current characteristic of one or more analytes in the sample, determining the amount of analyte in the sample in an apparatus according to the second aspect of the invention.

The following non-limiting examples illustrate at least some embodiments of the invention:

### EXAMPLES

SWCNTs were first grown by aerosol CVD as discussed in detail by Kaskela, A et al. in Aerosol-Synthesized SWCNT Networks with Tunable Conductivity and Transparency by a Dry Transfer Technique. Nano Lett. 2010, 10 (11), 4349-4355. https://doi.org/10.1021/n1101680s. and by Moisala A et al. in Single-Walled Carbon Nanotube Synthesis Using Ferrocene and Iron Pentacarbonyl in a Laminar Flow Reactor. Chem. Eng. Sci. 2006, 61 (13), 4393-4402. https://doi.org/10.1016/j.ces.2006.02.020., the methods of both of which are incorporated herein by reference and collected on a filter. The 18 × 26 cm SWCNT network was then press-transferred onto an A4 PET sheet and densified by spraying IPA from a spray bottle and dried with nitrogen. The SWCNT electrode made with the same process have previously been characterized in detail by Wester, N. et al. in Simultaneous Detection of Morphine and Codeine in the Presence of Ascorbic Acid and Uric Acid and in Human Plasma at Nafion Single-Walled Carbon Nanotube Thin-Film Electrode. ACS Omega 2019, 4 (18), 17726-17734. https://doi.org/10.1021/acsomega.9b02147. and by Wester, N. et al. in Single-Walled Carbon Nanotube Network Electrodes for the Detection of Fentanyl Citrate. ACS Appl. Nano Mater. 2020, acsanm.9b01951. https://doi.org/10.1021/acsanm.9b01951., the teachings of which are incorporated herein by reference. The press-transferred SWCNT had an optical transparency of 71.6% (550 nm) and sheet resistance of 73 Q/sq. To realize patterned electrodes, lines separating the electrodes were ablated with pulsed laser ablation.

To realize a reference electrode and reduce the resistance of the wire between the active electrode area and the contact pad silver lines were screen printed directly on top of the SWCNT layer (See FIG. 1, step 3). Silver contact pads were also fabricated in the same process. Finally, the whole A4 PET sheet was coated with Nafion with a slot die coater (Schneider Electric) at room temperature. For this process 5% Nafion solution (Sigma Aldrich) was diluted with ethanol (94.5 wt-%, Altia, Finland) to 2.5% before coating. The following slot die coating parameters were used: Coating width: 200.0 mm, Syringe dimeter: 22.0 mm, Pump rate: 1.2 ml/min, Wet film thickness: 15.0 µm, Speed 40 cm/min. The PET sheet was placed in the slotdie coater so that the electrodes were coated first and contact pads last. Prior to measurements, the electrode was covered with a PTFE film (Saint-Gobain Performance Plastics CHR 2255-2) with a prepunched 6 mm hole. For single measurements, however, this mask was not required as the laser ablated area around the electrodes was hydrophobic enough to keep the 40 µL drop in place during measurement. After slotdie coating with Nafion electrical isolation of the electrodes was tested with a multimeter for each test strip.

The thickness of the Ag reference electrode and the SWCNT/Nafion layer measured with scanning electron microscope (SEM). Before imaging, cross-sectional samples were prepared with focused ion beam (FIB) milling. Both FOB milling and SEM imaging were carried out with FEI Helios NanoLab 600 dual-beam system. Before milling the samples were coated with 100 nm gold by evaporation, to serve as a conductive coating protecting from beam damage during ion-milling and SEM imaging. The cross-sections milled with 16 kV acceleration voltage in rough milling and 280/460 pA currents. SEM imaging was carried out with 5-30 kV and low currents of 43-170 pA. The thickness of the silver lines was also measured with a profilometer (Dektak 6 M) over several places of the lines and over the reference electrode.

The cyclic voltammetry (CV) with Ru(NH₃)₆ in KCl and the potential measurements of the screen printed Ag pseudoreference electrode were carried out with a Gamry Reference 600 potentiostat in a conventional 50 ml glass electrochemical cell. A three-electrode setup with a Pt wire counter electrode and a Ag/AgCl[sat.] (+0.199 V vs SHE, Radiometer Analytical) reference electrode placed in a Luggin capillary was used to measure the potential of the Ag electrode, connected as the working electrode. For the CV measurements performed in the 50 ml cell the integrated electrodes of the test strips were connected. In these measurements a modified serialATA cable was used as connector.

All differential pulse voltammetry (DPV) and CV experiments with 40 µL drops were carried out with a PalmSens4 portable potentiostat. The strips were directly connected to a connector purchased from PalmSens, where 2 mm banana clips can be connected to any electrode of the connector. To study the susceptibility of the Ag reference electrode to the Cl⁻ concentration, KCl solutions with different concentrations were prepared by dissolving KCl (Merk Suprapur) in deionized water (18.2 MOhm-cm).

Morphine hydrochloride was obtained from the University Pharmacy, Helsinki, Finland. All other chemical were obtained from Sigma-Aldrich. For studying the electron transfer, 1 mM solution of the outer sphere redox probe Ru(NH₃)₆ were prepared in 1 M KCl and PBS. The paracetamol and interferent solutions were prepared in pH 7.4 phosphate-buffered saline (PBS) solution. Fresh stock solutions were prepared on each measurement day.

For the plasma measurements, expired human plasma (Octaplas AB, Sweden). The plasma samples were diluted with a 1:1 ratio by adding 1 ml plasma in 1 ml pH 7.4 PBS in an Eppendorf. The whole blood was obtained by finger-prick from a healthy volunteer and collected with 20 µL calibrated microcapillary tubes (Drummond Scientific Company, USA). The blood samples were then placed in 2 ml Eppendorf and diluted with 20 µL PBS. The plasma and whole blood samples with paracetamol were prepared by spiking the PBS used for dilution with twice the target PA concentration. To avoid clotting of the whole blood a new sample was obtained for each measurement. For each measurement a 40 µL drop was placed on the test strip with a micropipette. Because a slow increase in PA signal was observed with increasing accumulation time, an accumulation time of 2.5 min was used. Between each measurement the measured drop was wiped with tissue paper and rinsed with a PBS drop for 2.5 min before the next drop was placed on the test strip.

The overall thickness of the SWCNT/Nafion layer of the working electrode can be seen to be approximately 170 nm thick. A dark layer with a thickness of 65-75 nm between the SWCNT/Nafion layer and the Au coating (applied by electron beam deposition to protect the Nafion layer from beam damage during ion milling and SEM imaging) can also be observed likely due to Nafion. This result is in agreement with previous studies by us, e.g. Wester, N. et al. Simultaneous Detection of Morphine and Codeine in the Presence of Ascorbic Acid and Uric Acid and in Human Plasma at Nafion Single-Walled Carbon Nanotube Thin-Film Electrode. ACS Omega 2019, 4 (18), 17726-17734. https://doi.org/10.1021/acsomega.9b02147. and other groups, suggesting that the SWCNT are at least partially coated by Nafion. The cross-section of the Ag reference shows flat elongated Ag particles in the few µm in size range. Thicknesses between 5.9 to 7.2 µm were obtained for the cross-sections of the reference electrodes. Several measurements of the silver lines were also carried out with a contact profilometer giving thicknesses in the range of 5.5 to 7 µm. Due to the large roughness, a clear layer of Nafion cannot be discerned on top of the Ag particles.

Usually quasi-reference electrodes fabricated from silver suffer from drifting potentials during measurements, short life time, long run in times before the potential stabilizes and relatively short shelf life. While disposable test strips do not necessarily require long term stability, the run in time and potential drift during measurements can potentially cause problems. FIG. 3A shows the OCP potential vs an Ag/AgCl[sat] electrode of both the uncoated and Nafion coated screen printed Ag reference electrode in 0.1 M PBS solution. Both types of electrodes start at a potential of 84±1 mV. From FIG. 3A, it is, however, evident that the potential of the uncoated electrode drifts during the potential measurements. Despite this potential drift, the uncoated electrodes also reached a stable potential after approximately 1h. In contrast, the Nafion coated electrodes immediately shows a stable potential with no required run in time. One of the 4 Nafion coated electrodes was also measured for 7.5 h and gave an average potential of 84.78 mV ± 0.35. At no point during the measurement did the potential change more than ±1 mV as the lowest and highest measured potentials were 84.07 and 85.39 mV, respectively. A long term stability study was also carried out, where a potential drop of less than 10 mV (9.85 mV) was observed over 7 days of immersion in PBS. This potential stability and drift rate is comparable to screen printed Ag/AgCl electrodes with much more complicated design with protective layers incorporating salt matrix (KCl). The electrode in this work immediately produces a stable potential and remains stable for up to 7 days. These measurements clearly show that the Nafion coated electrodes can be used for voltammteric measurements in point-of-cate applications without any preconditioning. Moreover, one of the 4 measured electrode strips was from a different batch that was stored under ambient conditions for approximately 1.5 years prior to measurement. This electrode also showed a stable potential of 84.42 ± 0.47 mV during a 3 h measurement, indicating excellent shelf life of the reference electrode without any packaging of the electrode.

The susceptibility to Cl⁻ concentration was studied by measuring the potential of the fabricated Ag reference electrode vs. a conventional Ag/AgCl electrode in KCl solutions with different concentrations. FIG. 3B shows the potential of the Ag reference electrode as a function of the logarithm of the Cl⁻ concentration. The potentials of both the electrodes depend on the Cl⁻ concentration. The Nafion coated Ag electrode showed linear dependence on the Cl⁻ concentration of the electrolyte with a slope of -33.9 mV/log[Cl⁻]. The uncoated electrode showed lower dependence on the Cl⁻ concentration. Despite the susceptibility toward Cl⁻ concentration, the Nafion coated electrode shows an immediately stable potential at all concentrations without any run-in time. These results, however suggests that the ionic strength of the electrolyte solution should be controlled.

The electron transport was studied with the outer sphere redox probe Ru(NH₃)₆. FIG. 3C shows the CV measurements with various scan rates in 1 mM Ru(NH₃)₆ in 1 M KCl. A peak potential separation (ΔEₚ) of 68.8 mV (scan rate: 100 mV/s) was obtained, indicating close to reversible electron transfer. The increasing peak potential separation with increasing scan rate (110 mV with 400 mV/s), however indicates quasireversible electron transfer. The uncompensated resistance values of 164.1 ±25.6 Ω were also measured for 6 electrodes in PBS solution.

In the linearization of paracetamol concentration measurements correlation coefficients of R²=0.9959, R²=0.9999 and R²=0.9984 were obtained for PBS, plasma and whole blood respectively, indicating that the obtained signal depends linearly on the paracetamol concentration in the wide linear range from 1 µM to 2 mM, covering the entire physiologically relevant concentration range. The limit of detection (LOD) was calculated as LOD=(3*σ)/S), where σ is the standard deviation of three measurements in blank PBS and S the sensitivity over the whole linear range. The LOD was determined separately for 4 electrodes and an average value of 0.819±0.265 µM. The highest LOD was 1.06 µM, still well below the required cut-off concentration for paracetamol poisoning. Most clinical laboratories use cut-offs of approximately 66.15 µM (10 mg/L). The results show that the developed test strip can easily quantitatively determine the blood paracetamol at these levels even after dilution with 1:1 ratio of PBS and taking into account the lower recovery in plasma and whole blood.

The mean relative standard deviations (RSD) of the oxidation currents over the whole linear range were 4.3, 7.0 and 10.0 % in PBS, plasma and whole blood, respectively. It should be noted that the used plasma and whole blood come from different individuals. It should further be noted, that the whole blood measurement were carried out on 3 separate days, at different times of the day. Due to the larger variation in plasma and whole blood measurements in FIG. 5, single determinations were carried out with 3 electrodes in plasma spiked with 1 mM PA. In these measurements a relative standard deviation of 4.0 % and a recovery of 75.7±0.22% were obtained. This suggests that some passivation of the electrode may occur with prolonged measurements in protein containing solutions. As the electrodes are intended for single point-of-care determinations, a recovery test with spiked whole blood samples at 3 different concentrations was also carried out with 3 electrodes at each concentration. The results of this recovery study are shown in Table 1 and show recoveries around 74 %.

TABLE 1

Recovery study in whole blood. Average of 3 determinations with 3 different electrodes

| Added | Found | Recovery % | RSD % (n=3) |
|---|---|---|---|
| 50 | 36.5 | 73.1 | 7.4 |
| 100 | 74.7 | 74.7 | 5.5 |
| 500 | 371.8 | 74.4 | 1.9 |

Protein bound fractions of 20-25% have been previously reported for paracetamol. The unbound fraction was also found to be independent of concentration in the clinically relevant concentration range. Similar results were also obtained in a recent report, where we found recoveries of 60 and 40% for morphine and codeine, respectively, with a Nafion coated SWCNT electrode. All these recoveries closely match those for previously reported unbound fractions. Banis et al. also concluded that only the free fraction of clozapine, a benzodiazepine, contribute to the measured electrochemical signal in BSA containing analyte solutions with a chitosan-based composite coated electrode. These results suggest that the electrodes coated with polymer membranes can be used directly determine the unbound PA fraction, without the need for time-consuming equilibrium dialysis.

As can be seen from Table 2, lower detection limits have been previously reported by several groups. Similarly, relatively wide linear ranges have also been reported in previous work. However, as is evident from the treatment nomogram, extreme sensitivity is not required. Moreover, all the works in Table 2 rely on time-consuming sample processing including, precipitation of proteins (blood samples) and considerable dilution, to reduce matrix effects. It should also be noted that works starting with serum or plasma also have carried out pre-treatment of the blood samples. In contrast, the assay developed in this work can be used for determination of the PA concentration from whole blood, only after diluting with equal part PBS and without precipitation of proteins in less than 5 min. Thus, the result of this work presents a much simpler system with clear reduction in required sample treatment and thus faster assay time.

While these results show the applicability of the developed sensor strip and the proposed assay for screening of PA poisoning, further research is required to show the applicability with real patient samples. Pharmacokinetic parameters will need to be evaluated from both venous and capillary finger-prick blood samples. With further development, to achieve higher sensitivity or further miniaturization of the electrodes, the required sample size could also be further reduced.

TABLE 2

Non exhaustive comparison of electroanalytical methods for detection of PA in biological matrices current from

| Electrode | Method | DL (µM) | Linear range (µM) | Biological matrix | Sample treatment |
|---|---|---|---|---|---|
| SWCNT-GNS/GC electrode | DPV | 0.038 | 0.05-64.5 | human serum | Centrifuge: 30 min, 4000 rpm |
| | | | | | Precipitation of proteins: 2 ml |
| | | | | | Acetonitrile in 2 ml serum |
| | | | | | Vortexing: 45 s |
| | | | | | centrifuging: 10 min, 10000 rpm |
| CuNPs/C60/MWCNTs/CPE | adsorptive stripping SWV | 0.000073 | 0.009-0.4 | Urine, plasma, serum | Blood: Centrifuge: 30 min (separate serum and plasma) |
| | | | | | Precipitation of proteins: Acetonitrile and centrifugation to precipitate proteins |
| | | | | | Unspecified volumes of spiked samples transferred into the vol-tammetric with unspecified volume of phosphate buffer of pH 6.8 |
| | | | | | Urine Diluted 4 times |
| The Pd/GO modified GCE electrode | DPV | 0.0022 | 0.005-0.5 | Urine | Urine: Diluted 50 times with 0.1 M PBS (pH 6.8) Spiking of 5 mL human serum treated with acetonitrile for protein precipitation, Centrifugation 4000 rpm for 15 min dilution from 0.01 M to 10-25 µM |
| | | | 0.5-80 | | |
| ERG/GCE | DPV | 1.2 | 5-800 | Human serum | |
| MWCNTs:graphite/GC electrode | SWV | 0.157 | 0.472-13.2 | Urine Plasma | 1 mL of 0.2 M NaOH + 0.8 mL sample, 3 min vortexing, Addition of 3 mL ethyl acetate, 3 min vortexing, Centrifugation 5 min 4500 rpm removal of organic phase repeated extraction |
| | | | | | Drying of ethyl acetate phase under steam of nitrogen at 60° C. |
| Dowex50wx2 and gold nanoparticles modified glassy carbon paste electrode | adsorptive stripping square wave voltammetry | 0.00471 | 0.0334 -42.2 | Urine, Human blood serum | reconstitution with 20 mL buffer solution Urine (as is?) 50 µL serum in 25 mL buffer solution filtering through a 0.22 µm PVDF syringe filter |
| AuNPs/MWCNT/GCE For | DPV | 0.03 | 0.09-35 | Blood serum | Serum 1: precipitation of proteins 0.8 ml acetonitrile to 1 ml sample, spiking 10 min centrifugation diluted to final volume of 25 ml |
| | | | | | Serum 2 1 ml sample diluted of final volume of 50 mL with BRBS (pH 6.0) |
| RG/Ni2O3-NiO modified GC electrode electrochemically reduced graphene oxide (ER-GO)/Nafion glassy carbon electrode (GCE)] graphene/platinum nanoparticles/nafion composite modified glassy carbon electrode | DPV | 0.02 | 0.4- 100 | Urine | Not started |
| | SWV | 0.025 | 0.4– 1–10 | Urine | 2 ml sample + 8 ml 0.1 M ammonia buffer |
| | Adsorptive stripping square wave voltammetry | 1.06 × 10-10 | 8.2 × 10-6-1.6 × 10-9 M | Urine, blood serum | Filtering, 0.22 blood µm PVDF syringe filter |
| SWCNT/Nafion | DPV | 0.819 | 1-2000 | Plasma Whole blood | Dilution 50 µL sample in 25 ml buffer 20 µL samples + PBS spiked with analyte |

To verify that the lower recoveries in plasma and whole blood is not due to fouling by proteins, the passivation of the electrode was studied. First 1 mM Ru(NH₃)₆ was measured in both PBS and human plasma. FIG. 6 shows no apparent passivation of the electrode when 1 mM Ru(NH₃)₆ is measured in PBS and diluted human plasma. This result is in line with a similar passivation study carried out with a Nafion coated SWCNT electrode in previous reports. The passivation was further studied in high concentrations of PA, by performing 10 consecutive DPV scans in plasma and whole blood with 1 mM PA. These measurements gave RSDs of 3.6% in whole blood and 4.3% in plasma. These RSDs are comparable with the repeatiblity of the single determinations in Table 1 and that of PA in PBS. Furthermore, the electrodes used to measure 50 µM PA in whole blood (see Table 1) were also used to measure 50 µM PA in PBS. After wiping away the whole blood, washing with a 40 µL drop of PBS and confirming background returns to that of a blank PBS, a mean peak current of 1.83 ± 0.09 µM was obtained for 50 µM PA. This represents a recovery of 101.7 %, indicating that there is no permanent fouling after whole blood measurements.

The lack of any matrix effect in the background currents in FIG. 4 shows that no appreciable interference is caused by endogenous substances present in the plasma and whole blood samples. Despite this, several other drugs may cause interference in the determination of paracetamol. For this reason, several drugs frequently taken in concomitant overdose with paracetamol were tested. The drugs were tested at concentrations well above what is expected in blood samples. In the cases where the tested substances were found to cause interference the tolerance limit was defined as the maximum concentration of the interferent that caused an error of less than 5% in the PA determination. FIG. 7 shows the DPV scan in the absence and presence of a NSAID mix with 100 µM Ibuprofen, naproxen and aspirin, 1 mM salicylic acid (the metabolite of aspirin), 1 mM nicotine, 1 mM amoxicillin and 1 mM caffeine. PA is often co-administered with opioids, such as tramadol and morphine. Opioids are also one of the group of drugs most frequently taken in concomitant overdose with paracetamol. Moreover, Nafion has been shown to accumulate cations, such as opioids, the interference of the two common opioids morphine, the active metabolite of codein and heroin, and o-desmethyltramadol (ODMT), the active metabolite of tramadol, was also studied. Both morphine and ODMT are cations under physiological conditions and have phenol functionalities. Especially morphine has been shown to oxidize close to the same potentials as PA. For this reason, these two opioids were also tested for interference.

From FIG. 7 it is evident that the NSAID mix, 1 mM salicylic acid, 1 mM amoxicillin, 1 mM nicotine and 1 mM caffeine did not cause more than 5 % interference in the signal of 50 µM PA. Much lower tolerance limits of 2.5 µM morphine and 10 µM o-desmethyltramadol were obtained. Despite the relatively low tolerance limits, these concentrations represent high concentrations compared to therapeutic concentrations. Even in fatal cases of morphine and tramadol poisoning, the concentrations remain below the tested concentrations at approximately 1.75 µM and 3.8 µM, respectively.

As can be seen from Table 1 the assay repeatability was assessed by measuring 3 electrodes in 3 different concentrations in the physiologically relevant concentration range. Relative standard deviations of 7.4, 5.5 and 1.9% were obtained at concentrations of 50, 100 and 500 µM, respectively. It should be noted that these results were obtained by drawing 20 µL finger prick whole blood, diluting with PA spiked PBS solution, and transferring the sample onto the test strip with a micropipette. The RSD values therefore represent the cumulative error from all these steps.

The shelf-life of the electrodes was also tested after storage under ambient conditions for 4 months. The same sensitivity was achieved indicating excellent stability. Similarly, similar behavior of the Ag reference electrode was also observed after storage of 1.5 years.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to one embodiment or an embodiment means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment. Where reference is made to a numerical value using a term such as, for example, about or substantially, the exact numerical value is also disclosed.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, that is, a singular form, throughout this document does not exclude a plurality.

### INDUSTRIAL APPLICABILITY

At least some embodiments of the present invention find industrial application in the medical profession. A mass production compatible fabrication process of a disposable electrochemical test strip for use in quantitative point-of-care determination of neutral analytes in suspected cases of overdose of said neutral analytes such as paracetamol is described. With this process highly conductive, well electrically isolated and patterned carbon-based electrodes are printed on substrates. Furthermore, a screen printed silver pseudo-reference electrode with excellent shelf life, with long term stability and short hydration times is produced. With this test strip low enough detection limits and wide enough linear range for determination of neutral analyte concentration in suspected poisoning is achieved. The strip is particularly useful in the detection and determination of concentration of paracetamol in cases of suspected paracetamol overdose and/or poisoning.

The developed test strip can be used a highly portable and fast point-of-care assay for screening of paracetamol poisoning.

ACRONYMS LIST

| | |
|---|---|
| PA | paracetamol |
| UA | uric acid |
| AA | ascorbic acid |
| MO | morphine |
| CO | codeine |
| PBS | phosphate buffered saline |
| DPV | differential pulse voltammetry |
| PET | polyethyleneterephthalate |

1. A disposable multilayer test strip comprising a substrate onto which is deposited an electrode assembly, the electrode assembly comprising:
a carbon-based working electrode,
a carbon-based counter electrode,
a pseudo-reference electrode, wherein the pseudo-reference electrode, the working electrode, and the counter electrode are arranged adjacent to each other in the same plane, and
contacts for contacting the electrodes directly to a voltage supply,
wherein the test strip further comprises a permselective membrane layer, wherein said electrodes of the electrode assembly are electrically separated from one another, and wherein said electrode assembly layer is positioned between the substrate and the permselective membrane layer, and wherein the permselective membrane has a structure adapted to allow passage of one or more electronically neutral analytes in a sample to be analysed across the permselective membrane to the electrode assembly.

2. The strip according to claim 1, wherein the substrate is selected from the group consisting of a polymer and glass.

3. The strip according to claim 1, wherein the carbon-based electrode comprises a member selected from the group consisting of amorphous carbon, diamond-like carbon, graphite, graphene, carbon nanotubes, and a mixture thereof.

4. The strip according to claim 1, wherein the pseudo-reference electrode comprises silver.

5. The strip according to claim 1, wherein the pseudo-reference electrode consists of silver.

6. The strip according to claim 1, wherein the permselective membrane comprises a material selected from the group consisting of Nafion, cellulose acetate, dialysis membranes, polyvinyl sulfonate, carboxymethyl cellulose, polylysine, overoxidised polypyrrole, and sulfonated polymers.

7. The strip according to claim 1, wherein the permselective membrane comprises one or more layers of a membrane material applied to the strip, wherein a stack of membrane material layers forms the permselective membrane.

8. The strip according to claim 1, wherein the permselective membrane has a thickness in the range of 50 to 400 nm.

9. An apparatus comprising:
a memory configured to store reference data; and
at least one processing core configured to:
process information from a multilayer test strip according to claim 1;
compare the information from the strip according to claim 1 to the reference data; and
draw conclusions on the information processed from the strip according to claim 1.

10. A method for the detecting electronically neutral analytes in a sample comprising the steps of:
providing a sample,
contacting the sample electrically with a working electrode and a counter electrode of an electrode assembly of a multilayer test strip,
changing voltage between the working electrode and counter electrode,
measuring a current between the working electrode and counter electrode in relation to the voltage applied between the working electrode and the counter electrode, and
detecting a change in current characteristic of one or more analytes in the sample.

11. A method for the detecting electronically neutral analytes in a sample comprising the steps of:
providing a sample,
contacting the sample electrically with the working electrode and the counter electrode of the electrode assembly according to claim 1,
changing voltage between the working electrode and counter electrode,
measuring a current between the working electrode and the counter electrode in relation to the voltage applied between the working electrode and the counter electrode, and
detecting a change in current characteristic of one or more analytes in the sample.

12. The method according to claim 10, wherein the voltage between the working electrode and counter electrode is scanned from scanned from -0.2 V to 0.8 V at a scan rate.

13. The method according to claim 12, wherein the scan rate is in the range of 5 - 1000 mV/s.

14. A method of manufacturing a multilayer test strip comprising the steps of:
providing an SWCNT network,
pressing the SWCNT network onto a substrate, to form carbon-based electrodes,
separating the electrodes by laser patterning,
screen printing silver to form a silver pseudo-reference electrode adj acent to a carbon-based working electrode and a carbon-based counter electrode,
screen printing silver contact pads onto each electrode, and
coating the electrodes with a permselective membrane layer, wherein the coating step coats the electrodes with a predetermined thickness of the permselective membrane.

15. The method according to claim 14 for manufacturing a multilayer test strip according to claim 1.

16. The strip according to claim 1, wherein the one or more electronically neutral analytes comprise a member selected from the group consisting of paracetamol, tetrahydrocannabinol (THC), alprazolam, lorazepam, and a general anaesthetic.

17. The method according to claim 10, wherein the one or more analytes comprise a member selected from the group consisting of paracetamol, tetrahydrocannabinol (THC), alprazolam, lorazepam, and a general anaesthetic.

18. The method according to claim 11, wherein the one or more analytes comprise a member selected from the group consisting of paracetamol, tetrahydrocannabinol (THC), alprazolam, lorazepam, and a general anaesthetic.
